(19) United States
(12) Patent Application Publication
(10) Pub. No.: US 2012/0165826 A1
(43) Pub. Date: Jun. 28, 2012

(54) Uterine Manipulator and Cutting Unit

(75) Inventor: Jacqueline Therese Rose Rhemrev-Pieters, Den Haag (NL)

(73) Assignee: Tavigny B.V., Den Haag (NL)

(21) Appl. No.: 13/304,517

(22) Filed: Nov. 25, 2011

Related U.S. Application Data
(63) Continuation of application No. PCT/NL2010/050305, filed on May 20, 2010.

(30) Foreign Application Priority Data
May 25, 2009 (NL) 2002922

(51) Int. Cl. A61B 17/42; A61B 17/32

(52) U.S. Cl. 606/119; 606/167

(57) ABSTRACT

A uterine manipulator comprising a mobilization rod comprising a central tubular body connected in an articulated manner at one end to a first control grip and at the other end to a screw and means for transmitting commands from said first control grip to said screw and a complementary unit comprising a sleeve suitable for being associated coaxially and slidingly with said central tubular body of said mobilization rod, for guiding its movement, wherein the complementary unit has a sectioning device positioned at a distal end of the sleeve, which sectioning device is selected from the group comprising a knife-blade and an ultrasonic cutter.

### CROSS-REFERENCE TO RELATED APPLICATIONS

This application is a continuation application of International Patent Application Serial No. PCT/NL20101050305, entitled “Uterine Manipulator and Cutting Unit”, filed on May 20, 2010, which is a continuation of Netherlands Patent Application Serial No, 2002922, entitled “Uterine Manipulator and Cutting Unit”, filed on May 25, 2009, and the specifications and claims thereof are incorporated herein by reference.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### COPYRIGHTED MATERIAL

Not Applicable.

### BACKGROUND OF THE INVENTION

1. Field of the Invention (Technical Field)

The present invention relates to a uterine manipulator comprising:
a mobilization rod comprising a central tubular body connected in an articulated manner at one end to a first control grip and at the other end to a positioning organ and means for transmitting commands from said first control grip to said positioning organ; and
a complementary unit comprising a sleeve suitable for being associated coaxially and slidingly with said central tubular body of said mobilization rod, for guiding its movement.

2. Description of Related Art

Such a uterine manipulator is known from WO 2008/136024 and may be used when executing a laparoscopic hysterectomy.

As is well known in the art the function of uterine manipulators is to help surgeons to visualize the peritoneal cavity and the uterus by laparoscopy during a check-up or diagnostic examination or during a surgical operation, such as hysterectomy.

It is known from US 2001/0021854 to provide a uterine manipulator with a monopolar or bipolar electrode, which is rotationally provided on a cupula forming part of the manipulator. Said electrode is used for making an incision in and cut the vaginal wall directly via the vagina, in order to increase safety and speed of this operation whereby the dissected uterus can be removed through the vagina. A first problem of the device known from US 2001/0021854 is that the use of a monopolar or bipolar electrode for making an incision and cut the vaginal wall is that surrounding tissue is damaged. In case of using a bipolar electrode the damage concerns at least approximately five milimeter tissue at both sides of the cut whereas when using a monopolar electrode the damage at both sides of the cut concerns at least approximately 10 mm of tissue. The increase in vaginal cuff dehiscence is believed to be mainly caused by an excessive cauterization of the tissue during the amputation of the uterus from the vagina when using a monopolar or bipolar electrode.

A further problem of the device known from US 2001/0021854 is that its construction is quite complicated due to the necessity to have the electrode be construed in a manner that it can rotate around the stationary cupula.

### BRIEF SUMMARY OF THE INVENTION

It is an object of the invention to alleviate said problems and to provide a uterine manipulator with which the making of an incision and a cut in the vaginal wall can be performed with increased accuracy and with very limited damage to surrounding tissue.

The uterine manipulator of the invention is to that end characterized by the features of one or more of the appended claims.

### BRIEF DESCRIPTION OF THE SEVERAL VIEWS OF THE DRAWINGS

The invention will hereinafter be further elucidated with reference to some exemplary embodiments of the uterine manipulator of the invention and with reference to the drawing. Although the disclosure and discussion offered hereinafter concern embodiments of the uterine manipulator of the invention, it is expressly pointed out that the invention is also embodied in a separate cutting unit which is essentially embodied similarly as the complementary unit forming part of the disclosed uterine manipulator as discussed in the following.

In the drawings:

FIG. 1 and FIG. 2 show a mobilization rod and a complementary together forming the uterine manipulator of the invention;

FIG. 3 shows a detail of the combined mobilization rod and complementary unit;

FIG. 4 shows a first preferred embodiment pertaining to the uterine manipulator of the invention, and

FIG. 5 shows a detail of the second preferred embodiment of the uterine manipulator of the invention.

Wherever in the figures the same reference numerals are applied these numerals refer to the same parts.

### DETAILED DESCRIPTION OF THE INVENTION

In a first aspect of the invention the uterine manipulator is characterized in that the complementary unit has a sectioning device positioned at a distal end of the sleeve, which sectioning device is selected from the group comprising a knife-blade and an ultrasonic cutter. Making use of a knife or an ultrasonic cutter, largely avoids the tissue damage that occurs with the device known from US 2001/0021854.

When the sectioning device is an ultrasonic cutter this cutter can simply be switched on or off depending on the needs during the surgical operation.

When the sectioning device is a knife blade, this knife blade is preferably arranged such that it is retractable or rotatable between an operational and an inoperational position. In this manner also unintended damage to the vaginal wall or other tissue can be effectively avoided when moving the manipulator into or out of the vagina.

It is further preferred that the distal end of the sleeve is provided with at least one light source. This has the advantage that during the execution of the surgical operation, which takes place while simultaneously the external wall of the uterus is viewed in the abdominal cavity by using a laparoscopic viewing instrument, the position of the sectioning device inside the vagina can be simply detected by looking at the light from the light source shining through the vaginal wall. This is a very effective way to easily determine where to start with the incision and how the cut through the vaginal wall should be further executed and damage to other bodily parts such as the bladder, ureter and the intestines be avoided.

The applicant points out that the invention is also em-bodied in a separate cutting unit comprising an elongated body having a sleeve suitable for being associated coaxially and slidingly with a central tubular body of a mobilization rod, for guiding its movement, having a sectioning device positioned at a distal end of the body, which sectioning device is a knifeblade or an ultrasonic cutter.

A preferable embodiment of this cutting unit has the feature that the sectioning device is a knifeblade and that said knifeblade is retractable or rotatable between an operational and an inoperational position.

It is further preferred that the cutting unit of the invention has at the distal end of the body at least one light source.

### DETAILED DESCRIPTION OF THE INVENTION

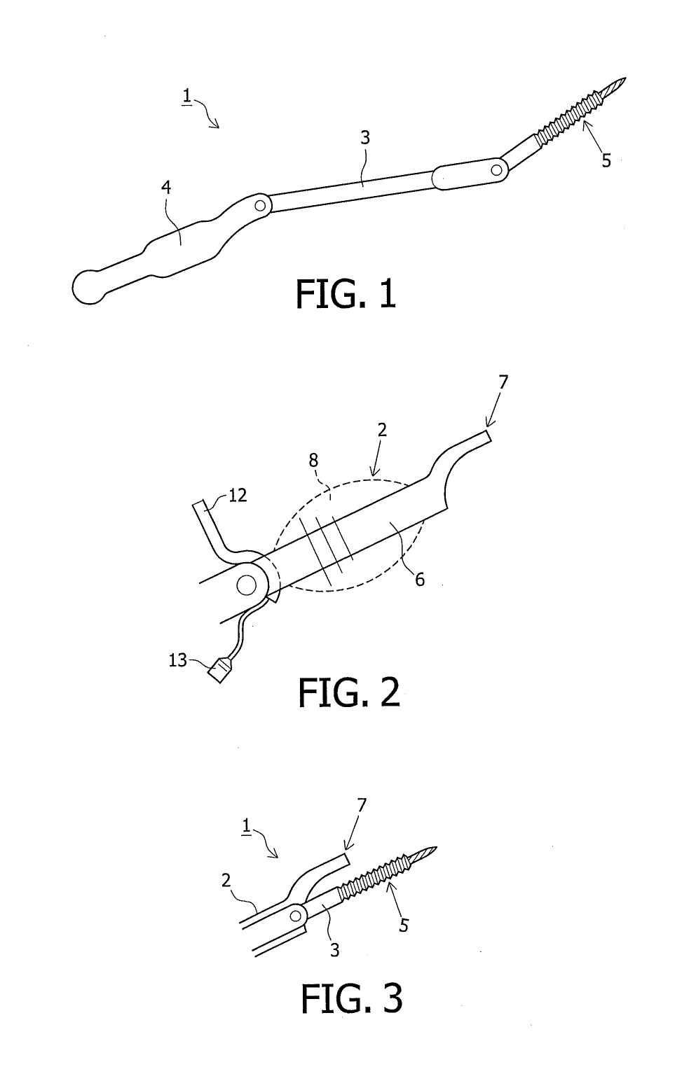

With reference first to FIG. 1 and FIG. 2 a mobilization rod 1 is shown comprising a central tubular body 3, which is connected in an articulated manner at one end to a first control grip 4 and at the other end to a positioning organ in the form of a screw 5. The first control grip 4 and the screw 5 are connected in a manner known per se by means for transmitting commands from the first control grip 4 to the screw 5. This is used in practice to manipulate the uterus in a desired position which is beneficial for carrying out a surgical operation.

The mobilization rod 1 as shown in FIG. 1 cooperates for that purpose with a complementary unit 2 as shown in FIG. 2 which comprises a sleeve 6, which is suitable for being associated coaxially and slidingly with the said central tubular body 3 of the mobilization rod 1 shown in FIG. 1. In this assembled position of the mobilization rod 1 and the complementary unit 2, the uterine manipulator may be inserted into the vagina up to the point where the screw 5 reaches a suitable position in the uterus for manipulating the uterus' position.

FIG. 2 shows further that the complementary unit 2 is provided with a second control grip 12, which can be used to rotate the sleeve 6 for bringing the distal end 7 of said sleeve 6 into a desired position with respect to the screw 5. Further, the complementary unit 2 is provided with a sealing means 8, for instance an inflatable balloon which can be inflated through a hose 13 for sealing off the vagina when carrying out the surgical operation in order to prevent the escape of gas, mostly CO₂, which is brought into the abdominal cavity for assisting the simultaneous laparoscopic viewing taking place through the abdominal wall.

For clarity purposes the assembled position of the mobilization rod 1 and the complementary unit 2 of the uterine manipulator is shown, in the detailed view of FIG. 3.

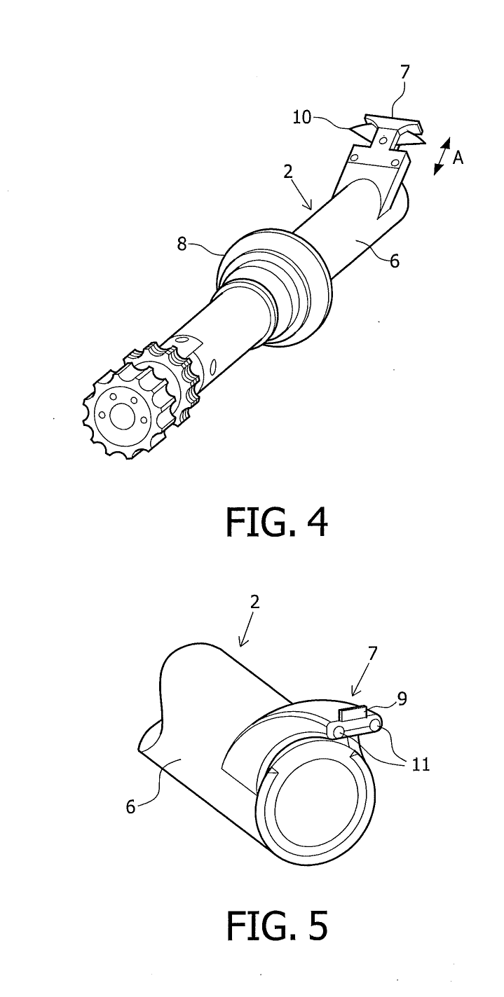

With reference further to FIG. 4 a first preferred embodiment of the complementary unit 2 of the uterine manipulator is shown, wherein at the distal end 7 of the sleeve 6 a knife blade 10 is provided which is retractable or—in the shown embodiment—rotatable according to the arrow A between an operational and an inoperational position. The knife blade 10 is shown in FIG. 4 in the operational position such that it can be used to make an incision and cut the vaginal wall in order to separate the uterus from the vagina.

FIG. 5 shows a detail of a second embodiment of the complementary unit 2 of the uterine manipulator of the invention, wherein the sectioning device positioned at the distal end 7 of the sleeve 6 is an ultrasonic cutter 9. The operation of this cutter 9 can be easily switched on and off from the proximal side of the uterine manipulator by a suitable controllable source of ultrasonic energy.

FIG. 5 also shows that the distal end 7 of the sleeve 6 is provided with at least one light source 11. In the shown embodiment there are two such light sources 11 which are located in the immediate vicinity or adjacent to the cutter 9 in order to assist the surgeon to decide where to initiate and make the cut in the tissue connecting the uterus to the vagina, by looking laparoscopically at the light shining through the vaginal wall into the abdominal cavity.

These light sources 11 provide a reliable indication where the sectioning device resides in the vagina. This makes it easy to avoid damage to neighboring intestines and other bodily parts, when making an incision into the vaginal wall in order to cut loose the uterus.

As mentioned above the invention is also embodied in a separate cutting unit having essentially the features of the complementary unit 2 that has been discussed here above. It is remarked though that such a separate cutting unit may be embodied with an elongated body that may be entirely or partly solid rather than be shaped as a sleeve, as is the case with the complementary unit forming part of the uterine manipulator of the invention.

What is claimed:

1. Uterine manipulator comprising:
a mobilization rod comprising a central tubular body connected in an articulated manner at one end to a first control grip and at the other end to a positioning organ and means for transmitting commands from said first control grip to said positioning organ; and
a complementary unit comprising a sleeve suitable for being associated coaxially and slidingly with said central tubular body of said mobilization rod, for guiding its movement; and
wherein the complementary unit has a sectioning device positioned at a distal end of the sleeve, which sectioning device is a knife-blade or an ultrasonic cutter.

2. Uterine manipulator according to claim 1, wherein the sectioning device is a knife-blade retractable or rotatable between an operational and an inoperational position.

3. Uterine manipulator according to claim 1, wherein the distal end of the sleeve is provided with at least one light source.

4. Cutting unit comprising an elongated body, having a sleeve, suitable for being associated coaxially and slidingly with a central tubular body of a mobilization rod, for guiding its movement, wherein it has a sectioning device positioned at a distal end of the body, which sectioning device is a knife-blade or an ultrasonic cutter.

5. Cutting unit according to claim 4, wherein the sectioning device is a knife-blade retractable or rotatable between an operational and an inoperational position.

6. Cutting unit according to claim 4, wherein the distal end of the body is provided with at least one light source.
